(19) United States
(12) Patent Application Publication
(10) Pub. No.: US 2024/0374836 A1
(43) Pub. Date: Nov. 14, 2024

(54) Injection Device and Supplementary Device

(71) Applicant: Sanofi, PARIS (FR)

(72) Inventor: Michael Helmer, Frankfurt am Main (DE)

(21) Appl. No.: 18/688,826

(22) PCT Filed: Sep. 5, 2022
(86) PCT No.: PCT/EP2022/074546

§ 371 Date: Mar. 4, 2024

(30) Foreign Application Priority Data
Sep. 7, 2021 (EP) 21315154.1

(51) Int. Cl. A61M 5/315; A61M 5/24

(52) U.S. Cl. CPC A61M 5/31568; A61M 2005/2403; A61M 2205/3306; A61M 2205/581; A61M 2205/582; A61M 2205/583

(57) ABSTRACT

An injection device includes: a housing configured to accommodate a medicament container filled with the medicament, a drive mechanism, and a fastener. The housing includes a sidewall with a recess. The drive mechanism is operably engageable with the medicament container for injecting the dose of the medicament. The fastener is arranged inside the recess and protrudes outwardly from an outside surface of the housing. The fastener includes a fastening structure complementary shaped to a counter fastening structure of a counter fastener of a supplementary device for fastening and/or fixing the supplementary device in a predefined position to the housing.

### CROSS REFERENCE TO RELATED APPLICATIONS

The present application is the national stage entry of International Patent Application No. PCT/EP2022/074546, filed on Sep. 5, 2022, and claims priority to Application No. EP 21315154.1, filed on Sep. 7, 2021, the disclosures of which are incorporated herein by reference.

### TECHNICAL FIELD

The present disclosure relates to an injection device with a fastener for a supplementary device and to respective supplementary device. The supplementary device is configured to assist a user in operating the injection device and/or for monitoring operation of the injection device by the user. In further aspect the disclosure relates to a drug delivery system comprising an injection device and a supplementary device.

### BACKGROUND

Drug delivery devices for setting and dispensing a single or multiple doses of a liquid medicament have a similar purpose as that of an ordinary syringe.

Drug delivery devices, such as pen-type injectors, are designed to meet a number of user-specific requirements. For instance, with patients suffering chronic diseases, such as diabetes, the patient may be physically infirm and may also have impaired vision. Suitable drug delivery devices especially intended for home medication can be designed to be robust in construction and easy to use. Furthermore, manipulation and general handling of the device and its components can be designed to be intelligible and easy understandable. Injection devices can provide setting and subsequent dispensing of a dose of a medicament of a variable size. Moreover, a dose setting as well as a dose dispensing procedure can be designed to facilitate an unambiguous operation of the drug delivery device.

### SUMMARY

Some drug delivery devices are design to provide a medicament for a patient suffering from a particular disease that may require a certain amount of the medicament to be either injected via a pen-type injection syringe or infused via a pump.

Some drug delivery or injection devices provide selecting of a dose of a medicament of variable size and injecting a dose previously set. Other injection devices provide setting and dispensing of a fixed dose. The amount of medicament to be injected in accordance to a given prescription schedule can always be the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, e.g., the medicament, has been used up.

In order to control and to supervise medication conducted by users or patients themselves it is an automated detecting and logging of a repeated and regular use of the drug delivery device can be provided. Especially for fixed dose injection devices typically offering a simple and rather effective approach for delivery of many drug therapies, recording of doses injected by a user can offer a significant advantage over a manual dose logging in terms of security and convenience.

Attachment of an add-on device to an injection device can provide an advantage, both in terms of providing a reliable monitoring of user operations conducted with the injection device as well as for providing a high degree of user acceptance. Attachment of an add-on device to the injection device can be designed to be easy to implement and to provide intuitive operation.

It can be advantageous to provide a well-defined fastening structure on the injection device for fastening and fixing the supplementary device to the injection device. The housing of the injection device may have to be specifically adapted and modified. A modification of a housing can involve the use of additional resources and may eventually require a complete redesign of the respective housing component.

An improved and simple way of providing a well-defined fastening mechanism for attaching and fixing a supplementary device to the housing of an injection device, such as a pen-type injector can be advantageous. The fastening mechanism can be designed to be easily integratable into an existing housing of an injection device. Moreover, the fastening mechanism can be designed to further enable an easy and straightforward mutual assembly and disassembly of the injection device and the supplementary device.

In a first aspect the present disclosure relates to an injection device for setting and injecting a dose of a medicament. The injection device comprises a housing configured to accommodate a medicament container filled with the medicament. The housing comprises a sidewall with at least one recess. The injection device further comprises a drive mechanism operably engageable with the medicament container for injecting the dose of the medicament.

The injection device further comprises a fastener arranged inside the recess and protruding outwardly from an outside surface of the housing. The fastener comprises a fastening structure which is complementary shaped to a counter fastening structure of a counter fastener of a supplementary device for fastening and/or for fixing the supplementary device in a predefined position and/or orientation to the housing of the injection device.

Typically, the fastener and the housing of the injection device are provided as separate components of the injection device that require mutual assembly in the course of manufacturing of the injection device. The fastener may be implemented as a device component providing not only a fastening function for the supplementary device but also providing a secondary function for the operation and/or general handling of the injection device, e.g., for setting and/or injecting of the dose of the medicament.

The fastener provides a mechanical fastening interface for the counter fastener of the supplementary device. In case there are provided numerous and differently configured supplementary devices for use with one and the same type of injection device there may be provided a suitable and dedicated fastener allowing and supporting mutual arrangement and fixing of only one supplementary device of a series of supplementary devices to the injection device. In this way, and since the fastener is provided as a separate piece to be fixed and assembled to or in the sidewall of the housing of the injection device, the injection device can be individually adapted with differently-shaped mechanical fastening structures as provided by different types of fasteners.

The fastener, which is configured for arrangement inside the recess of the sidewall of the housing of the injection device provides an individually reconfigurable fastening structure for fastening of the supplementary device to a housing of an injection device without the necessity to modify the housing as such. Hence, for different types of supplementary devices and/or for different types of mounting or fastening adapters, there may be provided always one and the same housing of the injection device.

For instance and by selecting one of a number of available fasteners and by arranging the respective fastener inside the recess of the sidewall of the housing of the injection device, the housing and hence the injection device can be respectively adapted for providing a mechanical fastening structure for a desired or intended type of a supplementary device.

According to another approach the fastener may provide a twofold function. It may not only provide a fastening structure for the counter fastening structure of the supplementary device but the fastener may further contribute to the overall design of the housing. With some examples the fastener may provide a kind of a label for the injection device. With other examples, the fastener covers or closes the recess in the sidewall of the housing, which recess may be implemented as a through recess extending entirely through the sidewall of the housing. Here, the fastener may close and/or seal the recess as provided in the sidewall of the housing.

With some other examples the fastener is at least in portions transparent or translucent and may provide an unobstructed view of a portion of a number sleeve or other dose size indicator located inside the housing of the injection device. Hence, the fastener may provide or may form a window enabling visibility and legibility of a number sleeve or any other dose size indicating indicator through the sidewall of the housing.

With some examples, the fastener is a device component of the injection device. It may contribute to the outer appearance and may influence the design of the injection device. With other examples it provides a window in the sidewall of the housing. With further examples the fastener may comprise an optical lens or a lens portion providing a magnified view of the number sleeve located inside the housing of the injection device. By providing an existing device component of an injection device with a fastener or fastening structure, the respective device component is now provided with a secondary function and purpose. When the fastener is implemented as a dose indicating window in the sidewall of the housing of the injection device it may provide a twofold function. In one aspect it provides a dedicated view of a portion of the number sleeve. In another aspect it provides a mechanical mount and hence a fastening interface for the counter fastening structure of the supplementary device.

According to another example the fastener comprises a base portion and an upper portion. The base portion is engaged with the recess of the sidewall of the housing and the upper portion protrudes outwardly from the outside surface of the housing. Typically, the fastening structure is exclusively located on or in the upper portion or in a transition zone between the upper portion and the base portion. The base portion is particularly shaped to engage with the recess as provided in the sidewall of the injection device. By way of the base portion the fastener itself can be suitably fastened to the housing, e.g., to the sidewall of the housing of the injection device.

With some examples the base portion and the recess may form or may contribute to a clip joint. Here, the fastener may be fastened to the housing of the injection device by way of respective snap or clip features.

With other examples the base portion may comprise a foot and a neck portion. The neck portion continues or extends into the upper portion. The neck portion is located between the upper portion and the base portion. In an assembly configuration the base portion may be located inside the housing of the injection device and may be in abutment with an inside surface of a side edge of the recess or through opening of the sidewall. The neck portion of the fastener May extend through the sidewall and the upper portion of the fastener may be entirely located outside the housing of the injection device.

The fastener may be provided as a single piece. The base portion and the upper portion and the optional neck portion may be integrally formed.

With further examples the fastener and the recess may be mutually joined or connected by way of a friction fit, a form fitting structure, by way of an adhesive and/or by way of welding.

With a series of differently configured fasteners the base portion may be implemented always in the same way and may not differ whereas the upper portion may be individually designed and configured for engagement with complementary and differently configured counter fastening structures of different supplementary devices.

According to another example the fastener comprises a fastening structure with at least a first side edge portion to engage with a first counter fastening portion of a counter fastening structure of the counter fastener. With some examples the housing is of elongated tubular shape. Here, the first side edge portion of the fastener or fastening structure extends along or is coaxial with the elongation of the housing. The first counter fastening portion of the counter fastening structure may be shaped accordingly. It may also extend along the longitudinal axis or longitudinal direction of the housing when the supplementary device is correctly assembled to the housing of the injection device.

According to a further example the fastening structure comprises at least a second side edge portion opposite to the first side edge portion. The second side edge portion is configured to engage with a second counter fastening portion of the counter fastening structure. An outside distance between the first side edge portion and the second side edge portion can correspond to an inside distance between the first counter fastening portion and the second counter fastening portion of the counter fastening structure.

In a final assembly configuration, in which the fastening structure is engaged with the counter fastening structure the first side edge portion may be in engagement with the first counter fastening portion and the second side edge portion may be in engagement with the second counter fastening portion. In other words, the oppositely located side edge portions of the fastening structure may be located or may be sandwiched between respective first and second counter fastening portions of the counter fastening structure of the supplementary device. In this way, and if the outside distance between the first and second side edge portion closely matches an inside distance between first and second counter fastening portions the counter fastening structure can be tightly and/or firmly secured and fixed to the fastening structure at least with regards to a direction extending from the first fastening structure to the second fastening structure.

With some examples and when the housing is of tubular shape and when the first side edge portion and the second side edge portion are located at a well-defined tangential offset from each other, mutual engagement between complementary shaped first and second counter fastening portions with the side edges of the fastening structure provides a fixing of the supplementary device to the housing of the injection device at least with regards to the tangential direction.

According to a further example the fastening structure comprises one a front edge portion and a rear edge portion to engage with a third counter fastening portion of the counter fastening structure. With some examples the front edge portion and/or the rear edge portion are located between the first and second side edge portions. With some examples the front edge portion extends between front ends of substantially parallel extending first and second side edge portions. With some examples the rear edge portion of the fastening structure connects the respective rear ends of first and second side edge portions.

With a tubular shape of the housing of the injection device the front edge and the rear edge form oppositely located longitudinal edges of the upper portion of the fastener that protrude from the outside surface of the housing. The front edge portion may face in a distal direction and the rear edge portion may face in a proximal direction.

The counter fastening structure may be provided with a third counter fastening portion to engage with at least one of the front edge portion and the rear edge portion of the fastening structure. Typically, the third counter fastening portion may get into longitudinal or axial abutment with at least one of the front edge portion and the rear edge portion. In this way, the counter fastening structure can be axially or longitudinally fixed to the fastening structure. In other words, by way of a mutual engagement of the third counter fastening portion with at least one of the front edge portion and the rear edge portion of the fastening structure there can be provided a longitudinal or axial stop, thus defining the longitudinal or axial position of the supplementary device relative to the housing of the injection device.

With some examples the third counter fastening portion extends between a longitudinal end of the first and second counter fastening portions. Here, the first and the second counter fastening portions may extend substantially parallel with respect to each other. The third counter fastening portion may extend there between. In effect, the first, second and third counter fastening portions may form a U-shaped counter fastening structure configured and shaped to receive a complementary-shaped rectangular or oval side edge of the upper portion of the fastener protruding from the outside surface of the housing.

According to a further example one of the fastening structure and the counter fastening structure comprises a groove. The other one of the fastening structure and the counter fastening structure comprises a protrusion or ledge portion to engage with the groove. Typically, the fastening structure and the counter fastening structure comprises mutually corresponding profiles or profile sections that complement each other to form a form fitting and/or friction fitting engagement between the fastener and the counter fastener.

The groove can extend along or parallel to the outside surface of the housing. The complementary shaped ledge portion or protrusion also extends along the elongation of the housing. The groove and the ledge portion or protrusion may become slidably engaged and may thus form a sliding guide between the fastening structure and the counter fastening structure. With some examples the groove or ledge portion extends along an entirety of at least one of the first side edge portion, the second side edge portion, the front edge portion and the rear edge portion of the fastening structure.

Likewise, the other one of the protrusion and the ledge portion may extend all along at least one of the first, the second and the third counter fastening portions of the counter fastening structure. The fastening structure may provide a rather tight and slack free mutual mechanical engagement between the fastening structure and the counter fastening structure of the housing and of the supplementary device, respectively. The fastening structure may be provide a secure, firm and failure safe mutual attachment and fixing of the supplementary device to the housing of the injection device can be provided.

With some examples the fastening structure comprises the groove and the counter fastening structure comprises the protrusion or ledge portion to engage with the groove.

According to another example the groove is provided in or extends along at least one of the first side edge portion, the second side edge portion, the front end portion and the rear edge portion.

With some examples the grooves may extend continuously from the first side edge portion into at least one of the front edge portion and the rear edge portion. The groove may extend further from at least one of the front edge portion and the rear edge portion into the second edge portion. With some examples the groove may extend all around the side edges of the upper portion of the fastener.

In this way, there can be provided a rather homogeneous groove structure along the edges of the upper portion of the fastener protruding outwardly from the outside surface of the sidewall of the injection device.

Providing the groove in the outwardly facing side edge of the fastener is beneficial to enable a rather smooth assembly of the fastener to the basis of the housing. With some examples, the fastener may be assembled from inside the housing. Here, in an assembly procedure the fastener may be introduced into the housing along a longitudinal direction and may be then aligned with the through recess provided in the sidewall of the housing. The fastener may by moved radially outwardly so that the upper portion thereof extends through the recess of the sidewall whereas the foot or a base portion of the fastener remains inside the housing of the injection device.

By providing the groove in or along an outside facing edge section of the fastener the cross-section of the fastener or the lateral dimensions of the fastener may remain less than or equal to respective inside dimensions of the recess as provided in the sidewall of the injection device.

According to another example the groove aligns with an outside surface of the housing. The fastening structure may provide a rather easy and intuitive mutual assembly and engagement between the groove and the protrusion or ledge portion of the counter fastening structure can be provided. The outside facing surface of the housing of the injection device may provide guidance for the protrusion or ledge portion of the counter fastening structure in order to smoothly engage and/or slide along the groove as provided on at least one of the first side edge portion, the second side edge portion, the front edge portion and the rear edge portion of the fastening structure.

According to another example at least one of the fastening structure and the counter fastening structure comprises a snap feature and the other one of the fastening structure and the counter fastening structure comprises a counter snap feature complementary shaped to the snap feature. Typically, the snap feature and the counter snap feature are arranged and configured such, that when the supplementary device reaches a final assembly configuration on or with the housing of the injection device the snap feature engages the counter snap feature, thereby securing and fixing the supplementary device to the housing.

With some examples, the snap feature is provided at a dedicated position of the groove and the counter snap feature is provided at a dedicated position of a complementary shaped ledge portion. There may be provided numerous snap features and complementary-shaped counter snap features on the first side edge portion and the second side edge portion and complementary on the first counter fastening portion and the second counter fastening portion. Alternatively or additionally, a snap feature may be provided on one of the front edge portion and the rear edge portion and a counter snap feature may be provided on the third counter fastening portion.

According to a further example the fastener comprises a body with a transparent window. With some examples the body of the fastener is made of a transparent material. The body may comprise a transparent plastic material, such as polycarbonate. The entire body may be transparent or only a portion thereof may be provided with the transparent window. The transparent window may be integrally formed with the body of the fastener. With some examples, the entire fastener and hence the entire body forms a window effectively covering or sealing the through recess as provided in the sidewall of the housing. Here, the fastener provides the secondary function of sealing, closing or encapsulating the interior of the housing of the injection device. Typically, the body answers or the entire fastener is manufactured as an injection molded plastic component. According to the examples described in the present disclosure, a desired shape and configuration of the fastener can be easily provided in a rather cost-efficient way.

With a further example of the injection device the drive mechanism comprises a number sleeve at least in sections arranged inside the housing. Here, at least a portion of the number sleeve overlaps with the recess or through recess as provided in the sidewall of the housing of the injection device. A portion of the number sleeve is hence visible through the transparent window of the fastener.

The window or fastener of the injection device can be made of a different material than the sidewall of the housing. In this way, and for providing a window in the sidewall allowing for a visual inspection of the number sleeve located inside the housing it is generally required to manufacture and to produce the housing and the fastener as separate components and to mechanically assemble these two components together in the course of assembly of the injection device.

The geometry of the transparent window can be modified to provide a fastening structure for the supplementary device and to integrate the fastening structure into the transparent window. The transparent window may provide a visibility of a portion of the number sleeve and may also serve as a mount or at least as a mounting aid for the supplementary device.

With some examples the window of the fastener comprises an optical lens. The optical lens is typically implemented as a magnifying lens. This enables a magnified or magnifying view of at least a portion of the number sleeve through the transparent window.

According to another aspect the present disclosure also relates to a supplementary device for attachment to an injection device as described above. The supplementary device comprises a housing and a counter fastener attached or fixed to the housing. The counter fastener comprises a counter fastening structure complementary shaped to the fastening structure of the injection device. Here and by way of the counter fastener, the supplementary device can be appropriated fixed and assembled to the housing of the injection device. Insofar, all features, effects and benefits as described above in connection with the injection device equally apply to the supplementary device.

The supplementary device may be implemented as a data logging device or as an add-on device suitable for monitoring operation of the injection device. The supplementary device may be operable and may be configured to record and to log repeated dose setting and dose injection procedures as conducted by the injection device. The supplementary device and in particular its counter fastener may be configured to releasably or to detachably connect to the housing of the injection device. This way, the supplementary device can be used with a series and with a multitude of different injection devices.

With the counter fastener complementary or correspondingly shaped to the fastener of the injection device, the supplementary device can be attached and fixed to the housing of the injection device always in a particular and predefined configuration, which may be required for a proper operation of the supplementary device, e.g., for measuring the size of a dose actually set and/or currently injected.

According to a further example the housing comprises an upper side and a bottom side opposite to the upper side. The counter fastening structure protrudes from the bottom side. With other examples, the counter fastening structure is provided as a recessed structure in the lower surface of the bottom side. When assembled to the housing of the injection device the bottom side faces towards the outside surface of the housing.

The counter fastening structure may be shaped and configured to slidably engage with the fastening structure as provided on the outside surface of the housing of the injection device. Mutual assembly of the supplementary device and the injection device may include a longitudinal sliding movement of the supplementary device relative to the injection device, e.g., along the longitudinal extension or longitudinal axis of the housing of the injection device.

According to a further example the counter fastening structure comprises at least a first counter fastening portion to engage with at least one of a first side edge portion, a second side edge portion, a front edge portion and a rear edge portion of the fastening structure.

With typical examples the counter fastening structure comprises a first counter fastening portion to engage with the first side edge portion. The counter fastening structure further comprises a second counter fastening portion to engage with the second side edge portion and further comprises a first counter fastening portion to engage with one of the front edge portion and the rear edge portion of the fastening structure of the fastener of the injection device.

With some examples the side edge portion of the fastener and the complementary shaped counter fastening portions of the counter fastening structure may form a sliding guide. A protrusion or ledge portion of one of the fastening structure and the counter fastening structure may slide inside the groove of the other one of the fastening structure and the counter fastening structure until a final assembly configuration of the supplementary device and the injection device is reached.

According to another example the supplementary device comprises a secondary fastener configured to clasp at least partially around an outside surface of the housing of the injection device. The secondary fastener may be located offset, e.g., longitudinally offset, and hence at a well-defined spatial distance from the counter fastening structure. The secondary fastener may be provided on the bottom side of the device housing. It may form or may comprise a clip to clasp at least partially around the outside surface of the housing of the injection device.

By way of the secondary fastener, the supplementary device can be attached and fixed to the housing of the injection device at least with regards to the radial direction. Mutual engagement of the fastener and the counter fastener provides at least one of a tangential and a longitudinal or axial fixing of the supplementary device to the housing of the injection device.

According to another example the supplementary device comprises a logic circuit, a signal generator connected to the logic circuit and at least one of a switch and a detector connected to the logic circuit. The at least one of the switch and the detector is operable to detect a correct assembly of the supplementary device to the housing of the injection device. Moreover, the logic circuit is operable to generate at least one of an audible, visual, or tactile signal via the signal generator in response to a detection of a correct or incorrect assembly of the supplementary device to the housing of the injection device.

The logic circuit and the signal generator may be operable to visually display a respective indicator, e.g., on an electronic display of the supplementary device thus indicating a correct or incorrect mutual assembly of the supplementary device and the housing of the injection device. Likewise, the signal generator may be operable to generate at least one of an acoustic or a haptic, hence vibrational signal by way of which at least one of a correct and an incorrect mutual assembly of the supplementary device and the injection device can be indicated to a user.

According to another aspect there is provided a drug delivery system. The drug delivery system comprises an injection device as described above and further comprises a supplementary device as described above. Insofar, all features, benefits, and effects as well as any synergistic effects obtained through a coupling and/or engagement between the injection device and the supplementary equally apply to the drug delivery system; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F (ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F (ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, examples of an injection device with a fastener for fixing and fastening of a supplementary device will be described in greater detail by making reference to the drawings, in which:

FIG. 1 shows a longitudinal cross-section through an injection device,

FIG. 2 shows a perspective view of the injection device according to FIG. 1,

FIG. 3 shows an isolated perspective view of a fastener for a supplementary device, wherein the fastener is integrated into a dosage window,

FIG. 4 shows a cross-section through a mutual assembly of the injection device and the supplementary device according to A-A as indicated in FIG. 9,

FIG. 5 shows a longitudinal cross-section B-B according to FIG. 9,

FIG. 6 illustrates one example of a counter fastening structure as provided on a bottom side of the supplementary device,

FIG. 7 shows the fastening structure and the complementary-shaped counter fastening structure in a pre-assembly configuration,

FIG. 8 shows the fastening structure and the counter fastening structure in a final assembly configuration,

FIG. 9 shows a perspective illustration of the injection device and the supplementary device prior or during mutual assembly,

FIG. 10 schematically illustrates a drug delivery system, wherein the supplementary device is fastened to the injection device,

FIG. 11 schematically illustrates another example of a fastener, and

FIG. 12 shows a block diagram of one example of the supplementary device.

### DETAILED DESCRIPTION

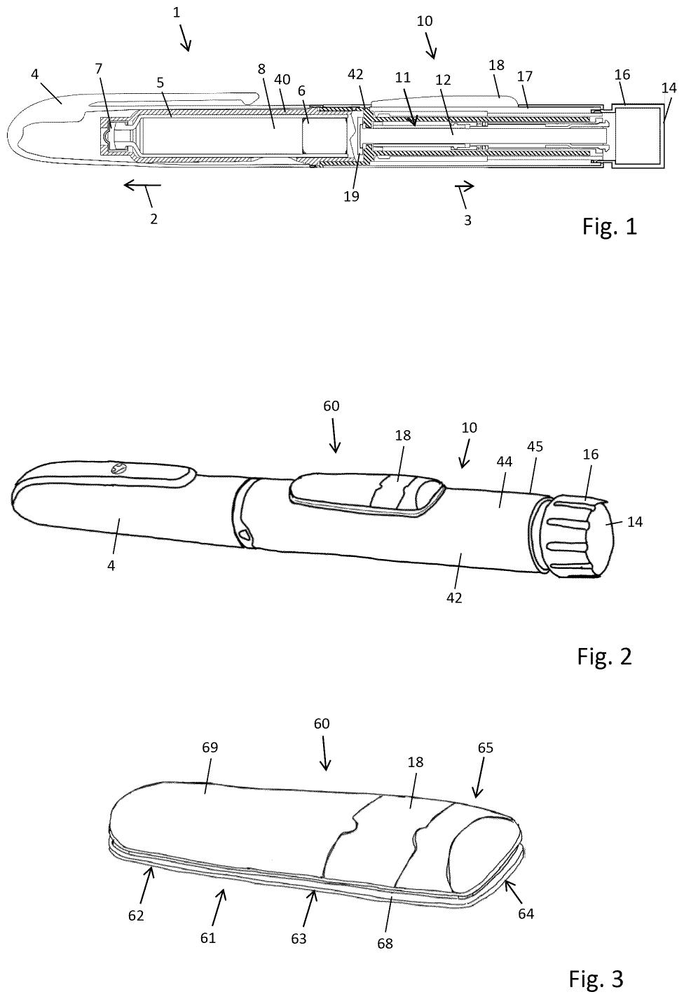

In FIG. 1 a longitudinal cross-section of one example of an injection device 1 is schematically illustrated. The injection device 1 is implemented as a pen-type injector. The injection device 1 may be implemented as a reusable device. It may be also implemented as a disposable device intended to be discarded, e.g., when the medicament container is empty.

The injection device 1 comprises a housing 10. The housing 10 comprises a first housing component 40, also denoted as a distal housing component and a second housing component 42 also denoted as a proximal housing component. The injection device 1 comprises a drive mechanism 11. The drive mechanism 11 comprises a piston rod 12 comprising a pressure piece 19 at a distal end. The piston rod 12 is configured to advance in a distal direction 2 and to exert pressure on a stopper 6 of a medicament container 5 located inside the first housing component 40.

The medicament container 5 comprises a tubular shaped barrel filled with the liquid medicament 8. Towards the distal end the medicament container 5 comprises an outlet provided with a seal 7. The seal 7 is configured to become penetrated by a double-tipped injection needle (not shown). Towards the proximal direction 3 the barrel of the medicament container 5 is sealed by the stopper 6. The stopper 6 is displaceable towards the distal direction 2 through the action of the distally advancing piston rod 12. In this way a dose of the medicament 8 can be expelled from the medicament container 5. The medicament container 5 may be implemented as a standard cartridge for pen-type injection devices 1.

The distal housing component 40 may be implemented as a cartridge holder configured to accommodate and to hold the medicament container 5. The injection device 1 may be further provided with a protective cap 4. The protective cap 4 effectively covers at least a major portion of the distal end of the injection device 1. It may provide protection for the first housing component 40 and may enclose a major part of the first housing component 40.

The drive mechanism 11 comprises numerous mechanically engaging components, such as a dose button 14, a dose dial 16 and a number sleeve 17. The dose dial 16 may be rotationally supported on the proximal housing component 42. It may be dialed or rotated by a user for setting of a dose of variable size. A user may depress the dose button 14 at the proximal end of the injection device 1 for initiating or for controlling a dose dispensing or dose expelling action of the drive mechanism 11.

The number sleeve 17 is at least partially visible through a window 18 or aperture in the second housing component 42. The number sleeve 17 comprises a consecutive sequence of numbers on an outside surface of its sidewall being indicative of a size of a dose currently set by the drive mechanism 11. The drive mechanism 11 may be implemented all mechanically. Here, a force required for advancing the piston rod 12 in the distal direction 2 is either provided by a user depressing the dose button 14 in distal direction 2. Alternatively, the drive mechanism 11 is provided with a mechanical energy storage, such as a drive spring. Mechanical energy stored in the mechanical energy storage may be released upon depressing of the dose button 14. Here, the force required to advance the piston rod 12 in distal direction 2 is entirely or at least partially provided by the mechanical energy storage. Examples of drive mechanisms 11 to be implemented with the injection device 1 can be found in WO 2014/033195 A1 or WO 2014/033197 A1 the entirety of which being incorporated herein by reference. The drive mechanism as disclosed in WO 2014/033195 A1 is a reusable drive mechanism. The drive mechanism as disclosed in WO 2014/033197 A1 is an example of a disposable drive mechanism being void of a reset function.

With other examples the drive mechanism 11 can be implemented electromechanically. Here, an electromechanical actuator may provide the force or torque required for advancing the piston rod 12 in distal direction 2.

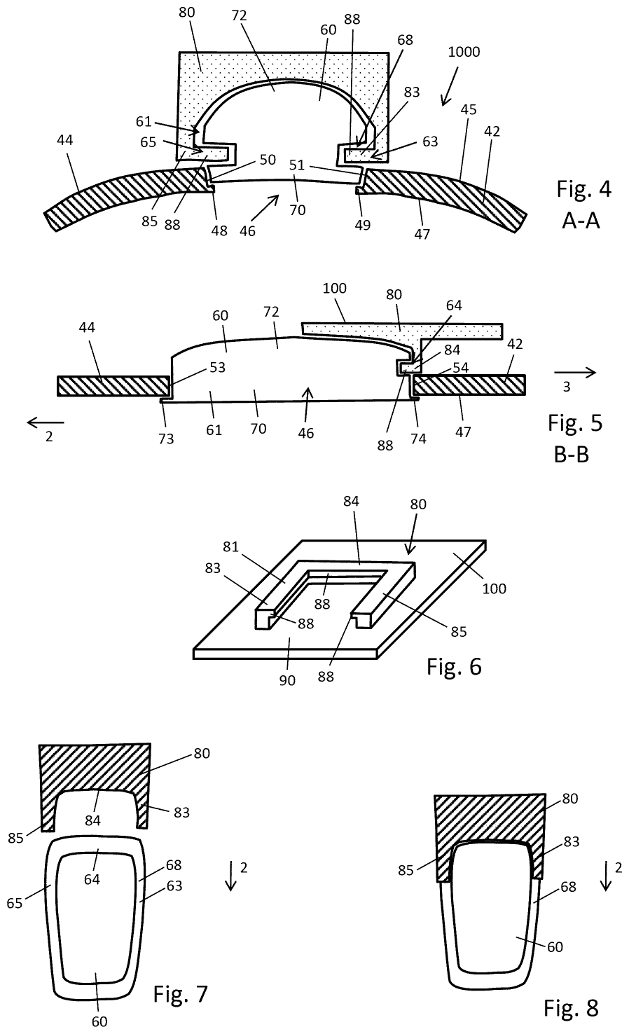

The proximal housing component 42 comprises a sidewall 44, which may be of substantially tubular shape. The injection device 1 further comprises a window 18 covering and/or closing a recess 46 as provided in the sidewall 44 as illustrated in FIG. 4. The recess 46 is implemented as a through recess. It provides unobstructed view of the number sleeve 17, which is typically provided with legible numbers or symbols on its outside surface. Typically, the window 18 is at least in sections transparent so as to provide legibility of the numbers of the number sleeve 17.

In the presently illustrated example, the window 18 comprises a body 69. The body forms or constitutes a fastener 60. In other words, there is provided a fastener 60 comprising a body 69, which body 69 in sections comprises a window 18 allowing for an unobstructed view through the body 69 and hence a view through the sidewall 44 from outside the injection device 1.

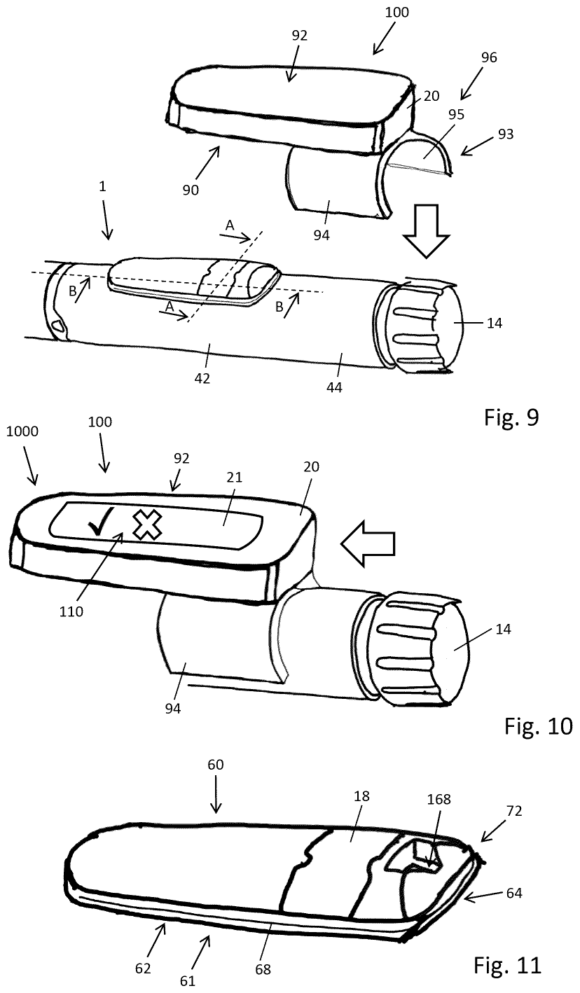

As becomes apparent from the illustrations of FIGS. 3-5 the fastener 60 not only provides a window 18 but also provides a fastening structure 61 by way of which a supplementary device 100 as illustrated in FIGS. 9 and 10 can be fastened and/or fixed to the housing 10 of the injection device 1.

With the drug delivery system 1000 as for instance shown in FIGS. 4 and 5 the fastener 60 comprises a base portion 70 and an upper portion 72. The base portion 70 is shaped and configured for insertion into the recess 46 of the sidewall 44 of the housing 10. The upper portion 72 protrudes from the outside surface 45 of the sidewall 44. Typically, it is the upper portion 72 that comprises a surrounding edge 62 that protrudes radially outwardly from the outside surface 45. The edge 62 comprises a first side edge portion 63 and a second side edge portion 65. The first and the second side edge portions 63, 64 extend along oppositely located side edges of the upper portion 72 of the body 69 of the fastener 60.

There is further provided a rear edge portion 64 interconnecting a rear end of the first side edge portion 63 and the second side edge portion 65. The first side edge portion 63 extends in longitudinal direction. It may extend coaxial to the distal direction or proximal direction 2, 3 as indicated in FIG. 1. The first side edge portion 63 extends at its proximal end into the rear edge portion 64. Opposite to the rear edge portion 64 there may be provided also a front edge portion extending between the distal end of the first and second side edge portions 63, 65.

At least one of the side edge portions and the rear and front edge portions 63, 64, 65 comprises a groove 68. The groove 68 forms or constitutes or belong to a fastening structure 61 for a complementary-shaped counter fastening structure 81 of a counter fastener 80 as provided on a bottom side 90 of a device housing 20 of the supplementary device 100. In the presently illustrated example, the fastening structure 61 comprises a somewhat circumferential groove 68 extending all over the first side edge portion 63, the rear edge portion 64 and the oppositely located second side edge portion 65. The groove 68 forms a slot or notch and is shaped to receive a complementary shaped protrusion or ledge portion 88 as provided on or by the counter fastening structure 81 of the counter fastener 80 of the supplementary device 100.

As illustrated in FIG. 6, the counter fastener 80 comprises a counter fastening structure 81 with a first counter fastening portion 83, a second counter fastening portion 85 and a third counter fastening portion 84. When mutually assembled as illustrated in FIGS. 7 and 8 the first counter fastening portion 83 is mechanically and positively and/or frictionally engaged with the first side edge portion 63. The third counter fastening portion 84 is engaged with the rear edge portion 64 and the second counter fastening portion 85 is in engagement with the second side edge portion 65.

As shown in FIG. 6, the counter fastening portions 83, 84, 85 form a kind of U-shaped structure which is open in distal direction 2 and which is shaped and configured to receive the proximally facing end of the upper portion 72 of the fastener 60 that protrudes from the outside surface 45 of the sidewall 44.

Each one of the counter fastening portions 83, 84, 85 comprises a somewhat longitudinal slat like profile. The counter fastening portions 83, 84, 85 comprises a somewhat L-shaped cross-section to engage with the groove 68 as provided along the outside facing edge 62 of the body 69 of the fastener 60. The counter fastening structure 81 is open towards the distal direction 2 and is shaped and configured to form at least one of a form fitting engagement and a friction fit with the edge 62 of the fastener 60 protruding outwardly from the outside surface 45 of the sidewall 44 of the housing 20.

The supplementary device 100 can be fixed to the housing 10 with regard to the tangential and/or circumstantial direction. When the inwardly extending ledges 88 of tangentially oppositely located first and second counter fastening portions 83, 85 engage the groove 68 as provided in the first and second side edge portions 63, 65 there will be also provided a mutual fixing between the supplementary device 100 and the housing 10 with regard to the radial direction.

In effect, the mutual sliding engagement between the first and second side edge portions 63, 65 with the complementary shaped ledge portions 88 of the first and second counter fastening portions 83, 84 prevents a rotation of the supplementary device 100 relative to the housing 10 with regard to the longitudinal axis of the housing 10 as an axis of rotation.

As further illustrated in FIG. 5 the distally protruding ledge portion 88 of the third counter fastening portion 84 is in longitudinal abutment or engagement with a complementary shaped section of the groove 68 in the region of the rear edge portion 64. By way of this longitudinal abutment or engagement the supplementary device 100 can be fixed to the housing 10 with regards to the longitudinal or axial direction.

The ledges 88 of any of the first, second and third counter fastening portion 83, 84, 85 may be integrally formed and may adjoin as illustrated in FIG. 6.

As further illustrated in FIG. 9 the supplementary device 100 also comprises a secondary fastener 93 extending from the bottom side 90 and comprising a fastening clip 96. The fastening clip 96 is configured and sized to clasp around at least a portion of the sidewall 44 of the proximal housing component 42. The fastening clip 96 may comprise two oppositely extending half shell portions 94, 95 adapted in shape to the outside surface 45 of the housing 10. As illustrated by the downward pointing arrow in FIG. 9 in a first step of assembly the supplementary device 100 may be fastened to the housing 10 by way of the fastening clip 96. Then and in a subsequence step of assembly as illustrated in FIG. 10 the supplementary device 100 may be subject to a distally directed sliding displacement, thereby bringing the fastening structure 61 and the complementary-shaped counter fastening structure 81 into mutual engagement. Once a final assembly configuration has been reached, which is e.g., illustrated in FIG. 8, the supplementary device 100 is firmly fixed to the injection device 1 thus forming or constituting a drug delivery system 1000.

In addition, or instead of a circumferential edge 62 provided with numerous side edge portions 63, 64, 65 there may be provided a groove 168 on or in the rear edge portion 64 of the upper portion 72 of the fastener 60 as illustrated in FIG. 11. Here, the supplementary device 100 may comprise a complementary shaped protrusion configured to engage with the groove or recess 168. The recess or groove 168 may comprise a somewhat tapered profile in longitudinal direction and/or in radial direction to provide a slack-free mutual fastening of the supplementary device 100 and the injection device 1.

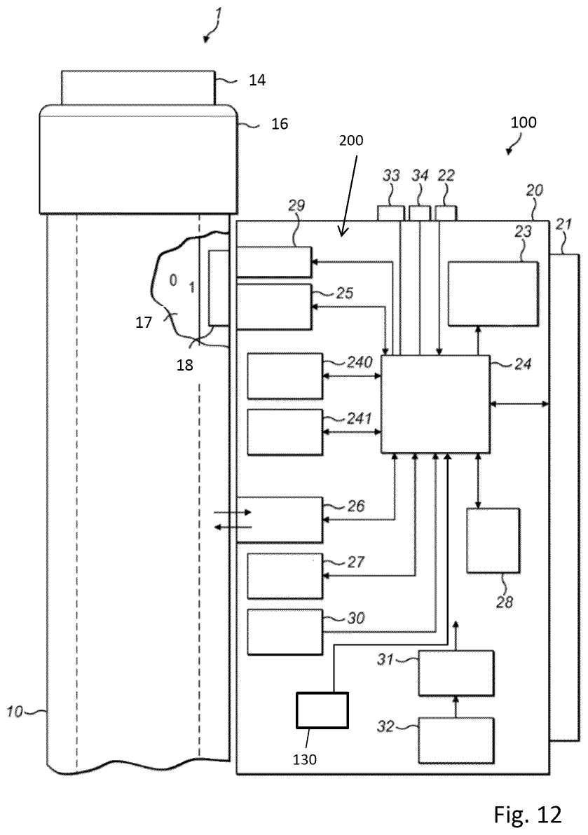

With some examples the supplementary device 100 is equipped with at least one of a switch 30 and a detector 130 as components of a logic circuit 200. By way of at least one of the switch 30 and the detector 130 a proper and correct installation or mounting configuration of the supplementary device 100 to the housing 10 can be detected. The logic circuit 200 typically comprises a processor 24 as described in further detail with reference to FIG. 12 below. In response to a detection of a correct mutual positioning of the supplementary device 100 to the housing 10 the processor 24 is operable to trigger a signal generator 23 to generate at least one of an audible, a haptic or a visual signal.

In FIG. 10, the display 21 as driven by the processor 24 is operable to provide at least one indicator 110 visually illustrating a correct or an incorrect positioning of the supplementary device 100 to the injection device 1. The display 21 is provided on an upper side 92 of the housing 20 of the supplementary device 100. It is located opposite to the bottom side 90. The switch 30 may be positioned or integrated e.g. in one of the first, the second and the third counter fastening portions 83, 84, 85 which engages the respective fastening structure 61 at the very end of a sliding assembly of the supplementary device 100 and the injection device 1.

The integration of a fastener 60 and hence of a fastening structure 61 into the window 18 of the injection device 1 is beneficial in numerous aspects. First of all, an existing component of the injection device 1, namely the window 18 and/or the body 69 comprising the window 18 is provided with a twofold function. On the one hand the body 69 seals and closes the recess 46 as provided in the sidewall 44 of the housing 10. Simultaneously, and on the other hand the body 69 and hence the fastener 60 provides a well-defined mounting or mechanical fastening interface for the supplementary device 100.

In case that different types of supplementary devices 100 each of which equipped with a different type of a counter fastening structure 81 should be provided for a respective variety of injection devices 1 the fastener 60 may provide a rather easy and straightforward mechanical encoding. Only by replacing the fastener 60 with a particular mechanical encoding by another fastener 60′ provided with another type of a mechanical coding, the variety of injection devices 1, all of which comprising one and the same proximal housing component 42 can be individually adapted for a dedicated use with only one particular supplementary device 100. The mechanical encoding is provided by the fastening structure 61. For providing the mechanical encoding of the injection device for a particular supplementary device it is hence not necessary to modify the proximal housing component 42. It is only required to replace a fastener 60 of a first type and provided with a first fastening structure 61 by another fastener 60′ of a second type provided with a fastening structure 61′ of a second type being incompatible with a counter fastening structure of the first type (not illustrated).

In the illustrations of FIGS. 4 and 5 the assembly of the fastener 60 in or through the recess 46 of the sidewall 44 is further illustrated. Here, oppositely located side edges 50, 51 of the recess 46 in the sidewall 44 are provided with a tangentially inwardly extending flange section 48, 49 at the level of the inside surface 47 of the sidewall 44. In effect, the base portion 70 and hence the side edges of the base portion 70 extending parallel to the side edge portions 63, 65 get in radial abutment with the flange sections 48, 49 when the fastener 60 is arranged inside the recess 46.

At the rear edge portion 64 and the oppositely located front edge portion the body 69 may comprise longitudinal extending projections 73, 74 as illustrated in FIG. 5. In the final assembly configuration the projections 73, 74 protruding longitudinally from the body 69 are in radial abutment with the inside surface 47 of the sidewall 44. The body 69 is typically made of a plastic material and exhibits a well-defined degree of elasticity. While the longitudinal side edges of the base portion 70 may be simply inserted from outside into the recess 46, the projections 73, 74 may snap under the longitudinal side edges 53 and 54 of the recess 46. In this way and since the base portion 70 effectively fills the void space of the through opening 46 the fastener 60 can be firmly fastened and fixed to the sidewall 44 of the housing component 42.

It should be noted that the illustrated example is by no way limiting. There may be many different ways of how to fix the fastener 60 and hence the transparent body 69 to the proximal housing component 42. When assembled to the sidewall 44 of the housing component 42 the groove 68 of the fastening structure 61 is effectively flush and/or is aligned with the outside surface 45 of the sidewall 44. This alignment and a substantially flush mutual arrangement of the groove 68 and the outside surface 45 is beneficial to provide a rather easy and intuitive sliding engagement of the ledge portions 88 of the counter fastening structure 81 with the groove 68 of the fastening structure 61.

FIG. 12 shows a schematic view of the supplementary device 100 in a state where it is attached to injection device 1 of FIG. 1.

With the device housing 20 of supplementary device 100, a plurality of components are comprised. These are controlled by a processor 24, which may for instance be a microprocessor, a Digital Signal Processor (DSP), Application Specific Integrated Circuit (ASIC), Field Programmable Gate Array (FPGA) or the like. Processor 24 executes program code (e.g. software or firmware) stored in a program memory 240, and uses a main memory 241, for instance to store intermediate results. Main memory 241 may also be used to store a logbook on performed ejections/injections. Program memory 240 may for instance be a Read-Only Memory (ROM), and main memory may for instance be a Random Access Memory (RAM).

In an example embodiment, processor 24 interacts with a first button 22, via which supplementary device 100 may for instance be turned on and off. A second button 33 is a communications button. The second button may be used to trigger establishment of a connection to another device, or to trigger a transmission of information to another device. A third button 34 is a confirm or OK button. The third button 34 can be used to acknowledge information presented to a user of supplementary device 100. The buttons 22, 33, 34 may be any suitable form of user input transducers, for instance mechanical switches, capacitive sensors or other touch sensors.

Processor 24 controls a display 21 or display unit, which is presently embodied as a Liquid Crystal Display (LCD). Display unit 21 is used to display information to a user of supplementary device 100, for instance on present settings of injection device 1, or on a next injection to be conducted. The display 21 or display unit may also be embodied as a touch-screen display, for instance to receive user input.

The processor 24 also controls an optical sensor 25, which may be embodied as an Optical Character Recognition (OCR) reader, that is capable of capturing images of the window 18 or dosage display, in which a currently selected dose is displayed (by way of numbers printed on the sleeve 17 contained in injection device 1, which numbers are visible through the window 18 or dosage display). OCR reader 25 is further capable of recognizing characters (e.g. numbers) from the captured image and to provide this information to processor 24.

The optical sensor 25 may be arranged on a side of the device housing 20 that faces towards the sidewall of the housing 10 of the injection device 1 when the counter fastener 80 of the supplementary device 100 is engaged with the fastener 60 of the injection device 1. The optical sensor 25 may be arranged relative to the counter fastener 80 such that when the counter fastener 80 is engaged with the fastener 60 the optical sensor 25 is aligned with the transparent window 18 of the fastener 60. This way, and when obtaining a predefined fastening configuration between the fastener 60 and the counter fastener 80 it can be provided that the optical sensor 25 is correctly aligned with the window 18.

Alternatively, the unit 25 of the supplementary device 100 may only be an optical sensor, e.g., a camera, for capturing images and providing information on the captured images to processor 24. Then processor 24 is responsible for performing OCR on the captured images.

The processor 24 also controls light-sources such as light emitting diodes (LEDs) 29 to illuminate the window 18, in which a currently selected dose is displayed. A diffuser may be used in front of the light-sources, for instance a diffuser made from a piece of acrylic glass. Furthermore, the optical sensor may comprise a lens (e.g., an aspheric lens) leading to a magnification (e.g. a magnification of more than 3:1).

The processor 24 further controls a photometer 26, that is configured to determine an optical property of the housing 10 of injection device 1, for example a color or a shading. The optical property may only be present in a specific portion of housing 10, for example a color or color coding of sleeve 17 or of an insulin container comprised within injection device 1, which color or color coding may for instance be visible through an opening or window in housing 10 (and/or in sleeve 17). Information on this color is then provided to processor 24, which may then determine the type of injection device 1 or the type of insulin contained in injection device 1 (e.g., first injection device with purple color and a second injection device with blue color). Alternatively, a camera unit may be used instead of photometer 26, and an image of the housing, sleeve or insulin container may then be provided to processor 24 to determine the color of the housing, sleeve or insulin container by way of image processing. Further, one or more light sources may be provided to improve reading of photometer 26. The light source may provide light of a certain wavelength or spectrum to improve color detection by photometer 26. The light source may be arranged in such a way that unwanted reflections, for example by the window 18, are avoided or reduced. In an example embodiment, instead of or in addition to photometer 26, a camera unit may be deployed to detect a code (for instance a bar code, which may for instance be a one- or two-dimensional bar code) related to the injection device and/or the medicament contained therein. This code may for instance be located on the housing 10 or on a medicament container contained in injection device 1, to name but a few examples. This code may for instance indicate a type of the injection device and/or the medicament, and/or further properties (for instance an expiration date).

The processor 24 further controls (and/or receives signals from) an acoustic sensor 27, which is configured to sense sounds produced by the injection device 1. Such sounds may for instance occur when a dose is dialed by turning dosage knob 16 and/or when a dose is ejected/injected by pressing injection button 14, and/or when a prime shot is performed. These actions are mechanically similar but nevertheless sound differently (this may also be the case for electronic sounds that indicate these actions). Either the acoustic sensor 27 and/or processor 24 may be configured to differentiate these different sounds, for instance to be able to safely recognize that an injection has taken place (rather than a prime shot only).

The processor 24 further controls a signal generator 23, e.g., implemented as an acoustic signal generator which is configured to produce acoustical signals that may for instance be related to the operating status of injection device 1, for instance as feedback to the user. For example, an acoustical signal may be launched by the signal generator 23 as a reminder for the next dose to be injected or as a warning signal, for instance in case of misuse. Acoustical signal generator may for instance be embodied as a buzzer or loudspeaker. In addition to or as an alternative to acoustical signal generator 23, also a haptic signal generator (not shown) may be used to provide haptic feedback, for instance by way of vibration.

The processor 24 controls a wireless unit 28, which is configured to transmit and/or receive information to/from another device in a wireless fashion. Such transmission may for instance be based on radio transmission or optical transmission. In some embodiments, the wireless unit 28 is a Bluetooth transceiver. Alternatively, wireless unit 28 may be substituted or complemented by a wired unit configured to transmit and/or receive information to/from another device in a wire-bound fashion, for instance via a cable or fiber connection. When data is transmitted, the units of the data (values) transferred may be explicitly or implicitly defined. For instance, in case of an insulin dose, always International Units (IU) may be used, or otherwise, the used unit may be transferred explicitly, for instance in coded form.

The processor 24 receives an input from a pen detection switch 30, which is operable to detect whether the pen 1 is present, e.g., to detect whether the supplementary device 100 is coupled to the injection device 1. A battery 32 powers the processor 24 and other components by way of a power supply 31.

The supplementary device 100 of FIG. 12 is thus capable of determining information related to a condition and/or use of injection device 1. This information is displayed on the display 21 for use by the user of the device. The information may be either processed by supplementary device 100 itself, or may at least partially be provided to another device (e.g., a blood glucose monitoring system).

### REFERENCE NUMBERS

1 injection device
2 distal direction
3 proximal direction
4 protective cap
5 medicament container
6 stopper
7 seal
8 medicament
10 housing
11 drive mechanism
12 piston rod
14 dose button
16 dose dial
17 number sleeve
18 window
19 pressure piece
20 device housing
21 display
22 button
23 signal generator
24 processor
25 optical sensor
26 photometer
27 acoustic sensor
28 wireless unit
29 light emitting diode
30 pen detection switch
31 power supply
32 battery
33 button
34 button
40 housing component
42 housing component
44 sidewall
45 outside surface
46 recess
47 inside surface
48 flange section
49 flange section
50 side edge
51 side edge
53 side edge
54 side edge
60 fastener
61 fastening structure
62 edge
63 side edge portion
64 rear edge portion
65 side edge portion
68 groove
69 body
70 base portion
72 upper portion
73 projection
74 projection
80 counter fastener
81 counter fastening structure
83 counter fastening portion
84 counter fastening portion
85 counter fastening portion
88 ledge portion
90 bottom side
92 upper side
93 secondary fastener
94 half shell portion
95 half shell portion
96 fastening clip
100 supplementary device
110 indicator
130 detector
168 groove
200 logic circuit
240 program memory
241 main memory
1000 drug delivery system

1-19. (canceled)

20. An injection device for setting and injecting a dose of a medicament, the injection device comprising:
a housing configured to accommodate a medicament container containing the medicament, the housing comprising a sidewall with a recess;
a drive mechanism operably engageable with the medicament container for injecting the dose of the medicament; and
a fastener arranged inside the recess and protruding outwardly from an outside surface of the housing, wherein the fastener comprises a fastening structure complementary shaped to a counter fastening structure of a counter fastener of a supplementary device for fastening and/or fixing the supplementary device in a predefined position to the housing.

21. The injection device of claim 20, wherein the fastener comprises a base portion and an upper portion, wherein the base portion is engaged with the recess and wherein the upper portion protrudes outwardly from the outside surface of the housing.

22. The injection device of claim 21, wherein the recess is a through recess extending entirely through the sidewall of the housing.

23. The injection device of claim 22, wherein the fastener closes and/or seals the recess.

24. The injection device of claim 21, wherein the base portion is located inside the housing and is in abutment with an inside surface of a side edge of the recess of the sidewall.

25. The injection device of claim 21, wherein the base portion comprises a foot and a neck portion, wherein the neck portion is located between the upper portion and the base portion.

26. The injection device of claim 20, wherein the fastener comprises a fastening structure with at least a first side edge portion to engage with a first counter fastening portion of a counter fastening structure of the counter fastener.

27. The injection device of claim 26, wherein the fastening structure comprises at least a second side edge portion opposite to the first side edge portion to engage with a second counter fastening portion of the counter fastening structure.

28. The injection device of claim 27, wherein the fastening structure comprises one of a front edge portion and a rear edge portion to engage with a third counter fastening portion of the counter fastening structure.

29. The injection device of claim 28, wherein one of the fastening structure and the counter fastening structure comprises a groove and wherein the other one of the fastening structure and the counter fastening structure comprises a protrusion or ledge portion to engage with the groove.

30. The injection device of claim 29, wherein the groove is provided in or extends along at least one of the first side edge portion, the second side edge portion the front edge portion and the rear edge portion.

31. The injection device of claim 29, wherein the groove aligns with an outside surface of the housing.

32. The injection device of claim 20, wherein the fastener comprises a body with a transparent window.

33. The injection device of claim 32, wherein the drive mechanism comprises a number sleeve at least partially arranged inside the housing and wherein a portion of the number sleeve is visible through the transparent window of the fastener.

34. A supplementary device for attachment to an injection device, the supplementary device comprising:
a device housing; and
a counter fastener attached and/or fixed to the device housing and comprising a counter fastening structure complementary shaped to a fastening structure of the injection device, wherein the injection device comprises:
a housing configured to accommodate a medicament container containing a medicament, the housing comprising a sidewall with a recess,
a drive mechanism operably engageable with the medicament container for injecting a dose of the medicament, and
a fastener arranged inside the recess and protruding outwardly from an outside surface of the housing, wherein the fastener comprises the fastening structure complementary shaped to the counter fastening structure of the counter fastener of the supplementary device for fastening and/or fixing the supplementary device in a predefined position to the housing.

35. The supplementary device of claim 34, wherein the device housing comprises an upper side and a bottom side opposite to the upper side and wherein the counter fastening structure protrudes from the bottom side or is integrated in the bottom side.

36. The supplementary device of claim 34, wherein the supplementary device comprises a secondary fastener configured to clasp at least partially around an outside surface of the housing of the injection device.

37. The supplementary device of claim 34, further comprising:
a logic circuit;
a signal generator connected to the logic circuit; and
at least one of a switch and a detector connected to the logic circuit and operable to detect a correct assembly of the supplementary device to the housing of the injection device, wherein the logic circuit is operable to generate at least one of an audible, visual or tactile signal via the signal generator in response to a detection of a correct assembly of the supplementary device to the housing of the injection device.

38. A drug delivery system comprising:
an injection device comprising:
a housing configured to accommodate a medicament container containing a medicament, the housing comprising a sidewall with a recess,
a drive mechanism operably engageable with the medicament container for injecting a dose of the medicament, and
a fastener arranged inside the recess and protruding outwardly from an outside surface of the housing, wherein the fastener comprises a fastening structure complementary shaped to a counter fastening structure of a counter fastener of a supplementary device for fastening and/or fixing the supplementary device in a predefined position to the housing; and
a supplementary device comprising:
a device housing; and
a counter fastener attached and/or fixed to the device housing and comprising the counter fastening structure complementary shaped to the fastening structure of the injection device.

39. The drug delivery system of claim 38, wherein the housing comprises an upper side and a bottom side opposite to the upper side and wherein the counter fastening structure protrudes from the bottom side or is integrated in the bottom side.
